(19) United States
(12) Patent Application Publication
(10) Pub. No.: US 2010/0101568 A1
(43) Pub. Date: Apr. 29, 2010

(54) Inhalation Anesthetic Device

(75) Inventors: Hyun-Chul Park, Pohang-city (KR); Yeong-Ho Jang, Daegu (KR); Kyeng-Ho Hwang, Anyangshi (KR); Hun-Kee Lee, Pohang-shi (KR)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: POSTECH ACADEMY-INDUSTRY FOUNDATION, Pohang-city (KR)

(21) Appl. No.: 12/325,759

(22) Filed: Dec. 1, 2008

(30) Foreign Application Priority Data
Oct. 28, 2008 (KR) 10-2008-0105845

(51) Int. Cl. A61M 15/00

(52) U.S. Cl. 128/203.12; 128/203.15

(57) ABSTRACT

The present invention relates to an inhalation anesthetic device whereby a patient inhales an anesthetic gas and is smoothly anesthetized. The present invention provides an inhalation anesthetic device that includes an inflatable member that inflates when a gas flows therein, an inhaling pipe through which the gas passes and that is connected to the inflatable member, and a gas container that contains an anesthetic gas and is connected to the inhaling pipe. According to the present invention, a patient is smoothly anesthetized while inflating an inflatable member.

### CROSS-REFERENCE TO RELATED APPLICATION

This application claims priority to and the benefit of Korean Patent Application No. 10-2008-0105845 filed in the Korean Intellectual Property Office on Oct. 28, 2008, the entire contents of which are incorporated herein by reference.

### BACKGROUND OF THE INVENTION

(a) Field of the Invention

The present invention relates to an anesthesia device. More particularly, the present invention relates to an inhalation anesthetic device whereby a patient is smoothly anesthetized while inhaling an anesthetic gas.

(b) Description of the Related Art

Anesthesia for surgery is divided into general anesthesia and local anesthesia. Further, the general anesthesia is divided into intravenous anesthesia and inhalation anesthesia.

Intravenous anesthesia is used to bring about unconsciousness by injecting drugs into a patient's vein. The intravenous anesthesia has a merit that patients show less rejection and it is easy to perform without special anesthetic devices. In addition, the intravenous anesthesia is suitable for simple and short surgery.

Inhalation anesthesia is a means of making a patient unconscious by injecting a volatile anesthetic agent into the patient's airway by using a mask or by performing endotracheal intubation. Inhalation anesthesia is performed in most surgeries because of quick induction of anesthesia and recovery, and because of its capability for maintaining anesthesia regardless of the length of a surgery.

To induce inhalation anesthesia, an anesthetic mask must be in contact with the face of a patient such that the patient inhales an anesthetic gas. However, child patients especially fear wearing an anesthetic mask on the face, which leads to a problem of an unsmooth induction of anesthesia. Accordingly, there is a need for an anesthetic device that is capable of relieving a patient of fear and inducing smooth anesthesia.

The above information disclosed in this Background section is only for enhancement of understanding of the background of the invention and therefore it may contain information that does not form the prior art that is already known in this country to a person of ordinary skill in the art.

### SUMMARY OF THE INVENTION

The present invention has been made in an effort to provide an inhalation anesthetic device that is capable of inducing smooth anesthesia wherein a patient inhales an anesthetic gas while inflating an inflatable member.

An inhalation anesthetic device according to an exemplary embodiment of the present invention includes an inflatable member that inflates when a gas flows therein, an inhaling pipe through which a gas passes and that is connected to the inflatable member, and a gas container that contains an anesthetic gas and is connected to the inhaling pipe.

The inhaling pipe may include a connecting portion that is connected to the inflatable member and a breathing portion that is opposite the connecting portion.

A first check valve may be disposed at the inhaling pipe, and the first check valve may be disposed between the connecting portion and the breathing portion.

A pressure reducing valve may be disposed at the inhaling pipe, and the pressure reducing valve may be disposed between the first check valve and the connection portion.

The inhaling pipe and the gas container may be connected via a gas pipe, and the gas pipe may be connected to the inhaling pipe between the first check valve and the breathing portion. A second check valve may be disposed at the gas pipe.

The inflatable member may be a balloon and the inhaling pipe may be made of plastic.

According to the present invention, a patient is smoothly anesthetized while inflating an inflatable member. Further, a patient may overcome fear or horror of anesthesia in an operating room because the patient has played with and become familiar with an inhalation anesthetic device according to the present invention.

Further, an inhalation anesthetic device according to the present invention can be manufactured such that it has a simple structure and is disposable, avoiding a risk of bacterial or viral infection that might be caused by using reusable anesthetic masks.

### BRIEF DESCRIPTION OF THE DRAWINGS

FIG. 1 is a schematic view of an inhalation anesthetic device according to an exemplary embodiment of the present invention.

FIG. 2 is a pneumatic circuit diagram of the inhalation anesthetic device according to an exemplary embodiment of the present invention.

### DETAILED DESCRIPTION OF THE EMBODIMENTS

In the following detailed description, only certain exemplary embodiments of the present invention have been shown and described, simply by way of illustration. As those skilled in the art would realize, the described embodiments may be modified in various different ways, all without departing from the spirit or scope of the present invention. Accordingly, the drawings and description are to be regarded as illustrative in nature and not restrictive. Like reference numerals designate like elements throughout the specification.

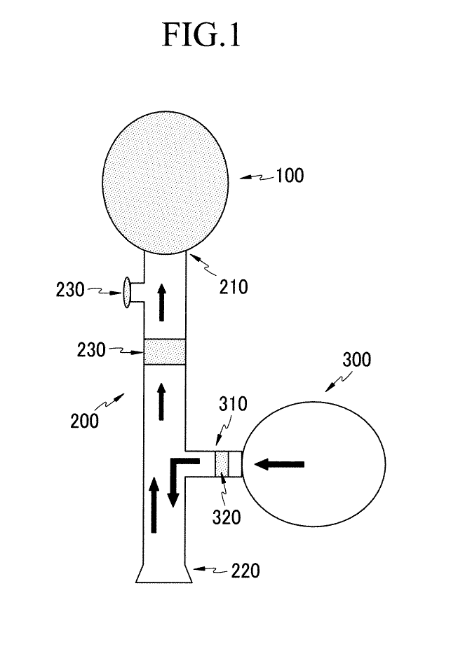

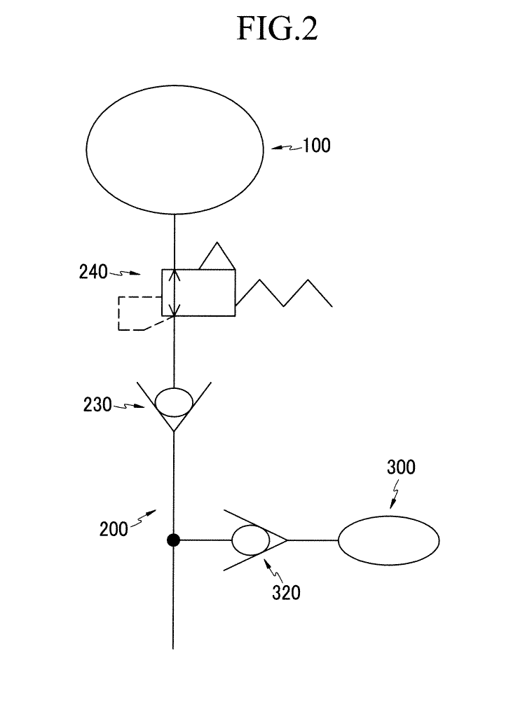

FIG. 1 is a schematic view of an inhalation anesthetic device according to an exemplary embodiment of the present invention, and FIG. 2 is a pneumatic circuit diagram of the inhalation anesthetic device according to an exemplary embodiment of the present invention.

Referring to FIG. 1 and FIG. 2, an inhalation anesthetic device according to an exemplary embodiment of the present invention includes an inflatable member 100 that inflates when a gas flows therein, an inhaling pipe 200 through which a gas passes and that is connected to the inflatable member 100, and a gas container 300 that contains an anesthetic gas. The gas container 300 ramifies from and is connected to the inhaling pipe 200.

The inflatable member 100 is made of a material that inflates when a gas flows into it from the outside. For instance, the inflatable member 100 may be a balloon that is made of rubber.

The inflating pipe 200 has a shape of a pipe through which a gas passes. The inflating pipe 200 includes a connecting portion 210 that is connected to the inflatable member 100 and a breathing portion 220 that is located opposite the connecting portion 210.

The connecting portion 210 is open toward the inflatable member 100 such that a gas may flow into the inflatable member 100. The inflatable member 100 is connected to the connecting portion 210 but is sealed to the outside such that the inflatable member 100 may be inflated with an inflowing gas. On the other hand, the breathing portion 220 is open to the outside such that a patient may inhale or exhale through the breathing portion 220.

Since the inhaling pipe 200 comes into direct contact with the mouth of a patient, the inhaling pipe 200 should be made of a non-toxic material. For instance, the inhaling pipe 200 may be made of plastic or glass.

A first check valve 230 and a pressure-reducing valve 240 are disposed at the inhaling pipe 200.

The first check valve 230 is located between the connecting portion 210 and the breathing portion 220. The first check valve 230 is a one-way valve; it opens when a gas flows from the breathing portion 220 to the connecting portion 210, but does not open when a gas flows from the connecting portion 210 to the breathing portion 220.

The pressure-reducing valve 240 is located between the first check valve 230 and the connecting portion 210. The pressure-reducing valve 240 discharges a gas inside the inflatable member 100 to the outside when the inflatable member 100 is over-inflated. That is, the pressure-reducing valve 240 helps reduce the pressure inside the inflatable member 100 and prevents the eruption of the inflatable member 100.

The gas container 300 contains an anesthetic gas and is connected to the inhaling pipe 200 via a gas pipe 310. That is, the anesthetic gas that is stored in the gas container 300 flows into the inhaling pipe 200 through the gas pipe 310. The gas pipe 310 is connected to the inhaling pipe 200 between the first check valve 230 and the breathing portion 220.

In addition, a second check valve 320 is disposed at the gas pipe 310.

The second check valve 320 is a one-way valve; it opens when the anesthetic gas flows from the gas container 300 to the inhaling pipe 200, but it does not open when an external gas flows from the inhaling pipe 200 to the gas container 300.

A more detailed description of the inhalation anesthetic device with the above-described configuration according to an exemplary embodiment of the present invention will be provided below.

First of all, a patient puts his/her mouth on the breathing portion 220 of the inhaling pipe 200 in order to inflate the inflatable member 100.

Then, the patient breathes in and collects breath with which he/she inflates the inflatable member 100. Here, the second check valve 320 opens as the anesthetic gas moves from the gas container 300 toward the inhaling pipe 200. During the above course, the anesthetic gas flows from the gas container 300 into the inhaling pipe 200, through the breathing portion 220, the patient's oral cavity, and consequently into the lungs.

Then, the patient inflates the inflatable member 100 by breathing out the breath he/she inhaled previously. Here, the first check valve 230 opens as the breath that is exhaled by the patient moves from the breathing portion 220 toward the inflatable member 100. During the above course, the exhaled breath flows through the inhaling pipe 200 into the inflatable member 100 and inflates the inflatable member 100.

Incidentally, even if the exhaled breath flows into the gas pipe 310, the exhaled breath cannot flow into the gas container 300. That is because the second check valve 320 does not open when a gas moves toward the gas container 300.

Then, the patient breathes in and collects breath in order to inflate the inflatable member 100 further. Here, even if the anesthetic gas flows again into the patient's body the air (the exhaled breath) already inside the inflatable member 100 does not flow into the patient's body. That is because the first check valve 230 prevents the air inside the inflatable member 100 from moving. That is, since the first check valve 230 is a one-way valve that opens only when air moves from the breathing portion 220 toward the inflatable member 100, it does not open when the air inside the inflatable member 100 moves from the inflatable member 100 toward the breathing portion 220.

With the above-described process repeated, the inflatable member 100 keeps inflating and the anesthetic gas keeps flowing into the patient's lungs. Accordingly, the patient is smoothly anesthetized during the course. When too much air flows into the inflatable member 100 the pressure-reducing valve 240 operates to discharge the air inside the inflatable member 100 to the outside.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

1. An inhalation anesthetic device comprising:
an inflatable member that inflates when a gas flows therein;
an inhaling pipe through which a gas passes and that is connected to the inflatable member; and
a gas container that contains an anesthetic gas and is connected to the inhaling pipe.

2. The inhalation anesthetic device of claim 1, wherein the inhaling pipe includes a connecting portion that is connected to the inflatable member and a breathing portion that is opposite the connecting portion.

3. The inhalation anesthetic device of claim 2, wherein a first check valve is disposed at the inhaling pipe.

4. The inhalation anesthetic device of claim 3, wherein the first check valve is disposed between the connecting portion and the breathing portion.

5. The inhalation anesthetic device of claim 4, wherein a pressure reducing valve is disposed at the inhaling pipe and the pressure reducing valve is disposed between the first check valve and the connection portion.

6. The inhalation anesthetic device of claim 4, wherein the inhaling pipe and the gas container are connected via a gas pipe, and wherein the gas pipe is connected to the inhaling pipe between the first check valve and the breathing portion.

7. The inhalation anesthetic device of claim 6, wherein a second check valve is disposed at the gas pipe.

8. The inhalation anesthetic device of claim 1, wherein a pressure reducing valve is disposed at the inhaling pipe.

9. The inhalation anesthetic device of claim 8, wherein the inhaling pipe and the gas container are connected via a gas pipe.

10. The inhalation anesthetic device of claim 9, wherein a second check valve is disposed at the gas pipe.

11. The inhalation anesthetic device of claim 1, wherein the inflatable member is a balloon.

12. The inhalation anesthetic device of claim 5, wherein the inhaling pipe and the gas container are connected via a gas pipe, and wherein the gas pipe is connected to the inhaling pipe between the first check valve and the breathing portion.

13. The inhalation anesthetic device of claim 12, wherein a
second check valve is disposed at the gas pipe.

14. The inhalation anesthetic device of claim 2, wherein a pressure reducing valve is disposed at the inhaling pipe.

15. The inhalation anesthetic device of claim 14, wherein the inhaling pipe and the gas container are connected via a gas pipe.

16. The inhalation anesthetic device of claim 15, wherein a
second check valve is disposed at the gas pipe
